(19) United States
(12) Patent Application Publication
(10) Pub. No.: US 2021/0170379 A1
(43) Pub. Date: Jun. 10, 2021

(54) Hydration of alpha-Pinene to obtain alpha-terpineol, using an ionic liquid as solvent, which is synthesized from a tertiary amine and an inorganic acid

(71) Applicant: Centro de Investigación en Materiales Avanzados, S.C., Chihuahua (MX)

(72) Inventors: Alfredo Aguilar Elguezabal, Chihuahua (MX); Manuel Roman Aguirre, Chihuahua (MX); Luis De la Torre Saenz, Chihuahua (MX); Lorena Alvarez Contreras, Chihuahua (MX)

(73) Assignee: Centro de Investigación en Materiales Avanzados, S.C., Chihuahua (MX)

(21) Appl. No.: 17/110,845

(22) Filed: Dec. 3, 2020

(30) Foreign Application Priority Data
Dec. 6, 2019 (MX) MX/A/2019/015134

(51) Int. Cl. B01J 31/02; C07C 29/09; B01J 27/16; B01J 27/02

(52) U.S. Cl. CPC B01J 31/0298; C07C 29/09; C07C 33/14; B01J 27/02; B01J 27/16

(57) ABSTRACT

An ionic liquid as a solvent in the hydration reaction of α-pinene to α-terpineol. The ionic liquid is obtained from the reaction of an amine and an inorganic acid. The use of the ionic liquid as solvent favors the selectivity towards the formation of α-terpineol and once the reaction product has been brought to room temperature, the organic phase can be physically separated from the inorganic one by decantation. The inorganic phase contains the ionic liquid, water and reaction catalyst and can be directly reused for a new reaction batch.

### OBJECT OF THE INVENTION

The present invention aims to use ionic liquids obtained from tertiary amines and inorganic acids as solvents in the hydration of α-pinene to α-terpineol. This invention has the advantage over other reported systems that the ionic liquid is low cost, it is prepared in a simple way, by a single step, and that the recovery of the products of the hydration of α-pinene is carried out by separating the two phases that are obtained by simple decantation. The ionic liquid does not require any treatment to be reused in the same reaction, as it remains stable.

### BACKGROUND

Terpineol is a product used in the perfume industry, in cosmetics, as a base for cleaners, detergents and surfactants for industrial applications. Although there are various sources of terpineol in a natural way, one of the most used ways to produce it, is the hydration of α-pinene. The transformation of α-pinene to terpineol has been the subject of numerous studies and there are several patents on the subject. Sapp (1953) developed a multi-step process that included the turpentine reaction (whose main component is α-pinene), using concentrated sulfuric acid (80% by weight), a second dilution step of the reaction mixture, centrifugation, washing with sodium carbonate, washing with water, and boiling with sulfuric acid (0.2 to 0.5 N) to obtain the crude terpineol. Herrlinger's patent (1959) describes the synthesis of αa-terpineol from α-pinene using sulfuric acid in water as a catalyst, in concentrations of 20 to 40%, and although it involves fewer steps than the aforementioned patent, this process has a lower yield, and it is necessary to use processes of high energy demand such as distillation, for the recovery of the product of interest. In addition, Herrlinger proposed that to improve the selectivity towards α-terpineol, some solvents such as hexane and para-cymene should be used, however, the drawback is that a process such as distillation must be used to recover said solvent. In Mexico, patents 126774 and 155026 describe a process for obtaining terpineol from sulfated turpentine that is very similar to that of the Sapp patent, since they consist of a process that includes the hydration of turpentine using sulfuric acid (25-35% by weight), separation and washing of the product with sodium carbonate, steam distillation, boiling with sulfuric acid and washing with soda.

In the scientific literature, the use of solid catalysts has been reported to facilitate the separation of the catalyst and the product, however, there is only a history of the use of an ionic liquid in this reaction, Liu et al. [2008] reported the use of 1-methyl-3-(3-sulfopropyl)-imidazolium dihydrogen phosphate ([HSO₃-pmim] H₂PO₄), however, the preparation of this ionic liquid is complex and involves high-cost reagents such as 1,3 propane sulfone, 1-methyl imidazole, in addition, requires ethyl acetate as a solvent both as a reaction medium and for washing 3-(1-methylimidazolium-3-yl) propane-1-sulfonate (MIM-PS), which is an intermediate product in the synthesis of ([HSO₃-pmim] H₂PO₄). The use of 1-methyl-3-(3-sulfopropyl)-imidazolium dihydrogen phosphate ([HSO₃-pmim] H₂PO₄) as ionic liquid concern a process that is unfavorable economically, and also from the point of view of caring for the environment.

This patent concerns a one-step ionic liquid synthesis method, with low-cost components and its use in the conversion of α-pinene to α-terpineol, allowing the separation of the products by a physical means, which is the decantation, since at room temperature two clearly distinguishable phases are formed.

### REFERENCES

Asis-Rahais, J., Procedimiento mejorado para la obtención de terpineol, Patente Mx 131,593 (1974).

Garcia-Leal A., Garcia Leal E., Procedimiento de fabricación de terpineol a partir de aguarrás sulfatado, Patente Mx 155,026 (1988).

Herrlinger, R., Production of alpha-Terpineol, U.S. Pat. No. 2,898,380 (1959).

Liu, S. W., Yu, S. T., Liu, F. S., Xie, C:X., Li, L., Ji, K. H., Reactions of α-pinene using acidic ionic liquids as catalysts, J. Molec. Catal. A:Chem. 279 (2008) 177-181.

Sapp, J. E., Production of terpineol U.S. Pat. No. 2,628,258 (1953).

### DETAILED DESCRIPTION OF THE INVENTION

The present invention describes a method to produce α-terpineol from α-pinene, using an ionic liquid as the reaction solvent that favors the selectivity and performance of the conversion of α-pinene to α-terpineol, with the advantage that once the reaction time has concluded, the ionic liquid is separated by decantation of the organic phase containing the terpineol and the by-products of the reaction, while the inorganic phase contains the ionic liquid with the catalyst used and water. Thus, the inorganic phase at bottom of reactor, can be used again for a new batch just adding α-pinene and the consumed water.

The ionic liquid that has been found to show the best results in the transformation reaction from α-pinene to α-terpineol is the product of the reaction of an inorganic acid with a tertiary amine, such as sulfuric acid with tripropylamine, that is tripropylamine acid sulfate. However, this patent is not limited to the use of this ionic liquid, since it was also found that other amines and other acids can be used, such as triethylamine, tributyl amine, triphenyl amine, tri isobutyl amine, and in the case of acids, phosphoric acid was also used, obtaining in all cases the formation of α-terpineol with a yield superior to that obtained by not using ionic liquid as a solvent.

For the preparation of the ionic liquid, an equimolar quantity of sulfuric acid of the highest available purity, preferably 99.5%, is added slowly and with constant stirring in a suitable container containing the required amount of the amine. Depending on the ambient temperature and the type of amine, it may be necessary to increase the temperature of the container, since the formation of the ionic liquid leads to a notable increase in viscosity. In order to eliminate the unreacted amine, when there is an excess of it, or the water present in the sulfuric acid, the mixture is left stirring between 90 and 110° C. until the emission of vapors is not observed.

The ionic liquid once synthesized can be stored until use. To carry out the reaction of pinene to terpineol, the ionic liquid is poured into the reactor, then the required amount of water is added., The catalyst is slowly poured (sulfuric acid or phosphoric acid) under stirring, verifying that the temperature does not rise uncontrollably.

Once the addition of the catalyst is complete, the reaction temperature is set. When the reactor is at the reaction temperature, pinene is added while maintaining stirring, and the system is kept under controlled temperature. Stirring is maintained for the required time to obtain the highest yield. Once the reaction time has ended, the reactor is allowed to cool to room temperature and later when the separation of the two phases (organic and inorganic) is clear, they are separated by decantation. The upper phase contains the unreacted pinene and the products formed from the pinene, while in the lower part, the mixture of water, catalyst and ionic liquid remains. The lower part of the mixture is transferred back to the reactor to carry out a new reaction. If necessary, the content of water is adjusted before the adding of new pinene load, to start another reaction.

### EXAMPLE 1 OF SYNTHESIS OF IONIC LIQUID AMINE-H₂SO₄

60 grams of tripropylamine (TPA) are placed in a suitable container (glass or stainless steel), and 41.6 grams of sulfuric acid (purity 98.5% by weight) are added slowly and under constant stirring. Once the mixture is finished, the ionic liquid is already formed. If necessary, the temperature of the container can be raised to 100° C., while maintaining the stirring. This heating ensures that the water present in any of the reagents used, or the excess of amine is eliminated, in case of variations in the purity of the reagents. With this procedure 101 grams of the ionic liquid are obtained. Hereinafter this ionic liquid is called TPAS.

### EXAMPLE 2 OF SYNTHESIS OF IONIC LIQUID AMINE-H₂SO₄

60 grams of tripropylamine (TPA) are placed in a suitable container (glass or stainless steel), and 48 grams of phosphoric acid (purity of 85.6% by weight) are added slowly and under constant stirring. Once the mixture is finished, the ionic liquid is already formed. If necessary, the temperature of the container can be raised to 100° C., while maintaining the stirring. This heating ensures that the water present in any of the reagents used, or the excess of amine is eliminated, in case of variations in the purity of the reagents. With this procedure 101 grams of the ionic liquid are obtained. Hereinafter this ionic liquid is called TPAP.

### EXAMPLE OF SYNTHESIS OF α-TERPINEOL FROM α-PINENE USING TPAS AS SOLVENT

24.1 g of TPAS, 12 g of water and 2 g of H₂SO₄ (purity of 98.5%) are fed into a reactor, as a last step 10.2 g of α-pinene are added. The mixture is heated to 70° C. and is kept with constant stirring at that temperature. The reaction is stopped after four hours of reaction and the contents of the reactor are cooled to room temperature. The reaction product separates into two phases and the upper part contains the reactants and products, while the lower part contains water, the ionic liquid and the catalyst. The analysis of the obtained product indicates that the conversion under these reaction conditions and time is 60% with selectivity towards α-terpineol of 41%.

### EXAMPLE OF SYNTHESIS OF α-TERPINEOL FROM α-PINENE USING IONIC LIQUID OBTAINED FROM TRIETHYLAMINE AS SOLVENT AND H₂SO₄ (TEAS)

19.9 g of TEAS, 6 g of water and 1 g of H₂SO₄ (98.5% purity) are fed into a reactor, as a last step 10.2 g of α-pinene are added. The mixture is heated to 60° C. and is kept with constant stirring at that temperature, the reaction is stopped after fifteen hours of reaction and the contents of the reactor are cooled to room temperature.

The reaction product separates into two phases and the upper part contains the reactants and products, while the lower part contains water, the ionic liquid and the catalyst. The analysis of the product obtained indicates that the conversion under these reaction conditions and time is 48% with a selectivity towards α-terpineol of 37%. The analysis of lower part shows that the ionic liquid contains water and the sulfiric acid.

### EXAMPLE OF SYNTHESIS OF α-TERPINEOL FROM α-PINENE USING AS SOLVENT THE IONIC LIQUID TPAS AND PHOSPHORIC ACID AS CATALYST

24.1 g of TPAS, 12 g of water and 4.58 g of H₃PO₄ (purity of 85.6%) are fed into a reactor, as a last step 10.2 g of α-pinene are added. The mixture is heated to 70° C. and is kept with constant stirring at that temperatura. The reaction is stopped after four hours of reaction and the contents of the reactor are cooled to room temperature.

The reaction product separates into two phases and the upper part contains the reactants and products, while the lower part contains water, the ionic liquid and the catalyst. The analysis of the product obtained indicates that the conversion under these reaction conditions and time is 25.2% with a selectivity towards α-terpineol of 47.7%. The analysis of lower part shows that the ionic liquid contains water and the phosphoric acid.

1-10 (canceled)

11. An ionic liquid comprising:
an amine; and
an acidic;
wherein the amine and the acid are reacted at equimolar proportions,
wherein the amine is a tertiaryamines selected from the group consisting of triethylamine, tripropylamine, triisopropylamine, tributylamine, tripentylamine, and mixtures thereof;
wherein the ionic liquid is a solvent for hydration reaction of α-pinene to α-terpineol.

12. The ionic liquid according to claim 11, wherein the acid is an inorganic acid selected from the group consisting of sulfuric acid, phosphoric acid, and mixtures thereof.

13. The ionic liquid according to claim 11, wherein for the hydration reaction of the α-pinene to α-terpineol, 0.5 to 2.5 moles of ionic liquid are used for each mole of pinene.

14. The ionic according to claim 11, wherein the hydration reaction of the α-pinene to α-terpineol further includes the use of 2-12 moles of water for each mole of pinene.

15. The ionic liquid according to claim 11, wherein the hydration reaction of the α-pinene to α-terpineol includes 0.10 to 0.40 moles of the acid used as catalyst for each mole of pinene.

16. The ionic liquid according to claim 11, wherein the ionic acid further includes an inorganic acid catalyst such as sulfuric acid or phosphoric acid.

17. A method for producing α-terpineol from α-pinene, the method comprising the steps of:
forming an ionic acid by reacting while stirring an amine with an acidic, a catalyst, and water; wherein the amine and the acid are reacted at equimolar proportions, wherein the amine is a tertiaryamines selected from the group consisting of triethylamine, tripropylamine, triisopropylamine, tributylamine, tripentylamine, and mixtures thereo;
using the ionic acid as solvent for a hydration reaction of α-pinene to α-terpineol.

18. The method of claim 17, wherein the reaction is performed at temperatures ranging from room temperature to 80° C.

19. The method according to claim 17, wherein the reaction times ranges from one hour to 36 hours.

20. The method according to claim 17, further including the step of:
cooling the ionic acid;
stopping the stirring;
wherein an upper and a lower phase are formed, the upper phase contains the α-terpineol and the lower phase contains the ionic liquid, the catalyst, and the water that did not react.

21. The ionic liquid according to claim 20, wherein the catalyst and liquid ionic are reused.
